(19) United States
(12) Patent Application Publication
(10) Pub. No.: US 2023/0071405 A1
(43) Pub. Date: Mar. 9, 2023

(54) Natural Killer Cells for Treatment of Coronavirus Infection

(71) Applicant: Sorrento Therapeutics, Inc., San Diego, CA (US)

(72) Inventor: Henry Hongjun Ji, Rancho Santa Fe, CA (US)

(73) Assignee: Sorrento Therapeutics, Inc., San Diego, CA (US)

(21) Appl. No.: 17/795,830

(22) PCT Filed: Jan. 27, 2021
(86) PCT No.: PCT/US2021/015332

§ 371 Date: Jul. 27, 2022

Related U.S. Application Data
(60) Provisional application No. 62/966,934, filed on Jan. 28, 2020.

(51) Int. Cl. A61K 35/17; C12N 5/0783; A61K 9/00; A61K 31/56; A61K 31/165; A61P 31/14

(52) U.S. Cl. CPC A61K 35/17; C12N 5/0646; A61K 9/0019; A61K 31/56; A61K 31/165; A61P 31/14; C12N 2501/2302; C12N 2501/2312; C12N 2501/2315; C12N 2501/2318; C12N 2501/2321

(57) ABSTRACT

The present disclosure provides a method for treating a subject having a coronavirus infection comprising administering to the subject at least one dose of a population of natural killer (NK) cells in an amount effective to reduce the coronavirus infection in the subject. In various embodiments, the NK cells administered to the subject are isolated NK cells, expanded and activated NK cells, or placental-derived NK cells.

This application claims the benefit of priority of U.S. Provisional Patent Application No. 62/966,934, filed Jan. 28, 2020, which is incorporated by reference herein in its entirety for all purposes.

### TECHNICAL FIELD

The present disclosure provides methods for treating a subject having a viral infection by administering at least one dose of a population of natural killer (NK) cells to the subject. The methods may be used to treat a coronavirus infection.

### INTRODUCTION

The coronavirus group of viruses causes diseases in birds and mammals, including humans. The diseases include respiratory infections and emetic infections which can range from mild to lethal. Coronaviruses are enveloped viruses having a positive-sense single-stranded RNA genome and a nucleocapsid of helical symmetry. The genome size of coronaviruses ranges from approximately 26 to 32 kilobases, which is believed to be the largest genome size for an RNA virus.

The name “coronavirus” is derived from the Latin corona and the Greek korone (e.g., “garland” or “wreath”), meaning crown or halo. The corona reference relates to the characteristic appearance of virions (the infective form of the virus) by electron microscopy, which have a fringe of large, bulbous surface projections creating an image reminiscent of a royal crown or of the solar corona. This morphology is created by the viral spike (S) peplomers, which are proteins that population the surface of the virus and determine host tropism. Proteins that contribute to the overall structure of all coronaviruses are the spike (S), envelope (E), membrane (M) and nucleocapsid (N). In the specific case of the SARS coronavirus, a defined receptor-binding domain (RBD) on S mediates the attachment of the virus to its cellular receptor, angiotensin-converting enzyme 2 (ACE2). SARS-CoV-2, first identified as a coronavirus causing the disease later called COVID-19 in Wuhan, China, in December of 2019, reached pandemic status in March of 2020 and to date is responsible for more than two million deaths worldwide. The existence of many asymptomatic carriers, pre-symptomatic patients, and patients with very mild symptoms present a huge challenge to infection control, as the transmission of SARS-CoV-2 from infected persons to susceptible groups has proven to be difficult to prevent. Multiple vaccines have recently been approved and vaccinations have begun, but the enormous number of people who have been infected worldwide and the high rate of transmission makes it unlikely that the spread of the virus will be brought down to acceptable levels for many months.

Further, multiple variants of the original SARS-CoV-2 virus are now in global circulation (cdc.gov/coronavirus/2019-ncov/transmission/variant.html), with the degree of protection against the variants afforded by currently available vaccines uncertain. With the number of actively infected individuals worldwide currently at about 25 million, it is likely that further variants will arise.

Acute Respiratory Distress Syndrome (ARDS), characterized by low levels of oxygen in the blood (PaO2/FiO2 ratio of less than 300 mmHg (≤39.9 kPa)) coincident with lung injury (ARDS Definition Task Force (2012) Acute respiratory distress syndrome: the Berlin definition. JAMA 307:2526-2533) can develop in response to viral infection, including infection with coronaviruses such as SARS, MERS, and SARS-CoV-2. Patients infected with SARS-CoV-2 that develop ARDS have a poor prognosis: a survey and meta-analysis of studies and reports from multiple countries (Hasan et al. ((2020) Expert Rev Respir Med. 14:11, 1149-1163) found that the overall pooled mortality estimate among 10,815 ARDS cases in COVID-19 patients was 39% (95% CI: 23-56%) stating: “The high mortality rate in COVID-19 associated ARDS necessitates a prompt and aggressive treatment strategy.”.

Natural Killer (NK) cells are part of the primary response to viral infection. As components of the innate immune system, NK cells have the unique ability to eliminate virus-infected cells due to the recognition of stress markers on the affected cells without relying on HLA-complex binding. For example, the NKG2D receptor expressed on NK cells recognizes induced proteins (e.g., the MIC and RAET1/ULBP proteins) which appear on the surface of stressed, malignant transformed, and virus infected cells. NK cells can also mediate antibody-dependent cell-mediated cytotoxicity (ADCC) by recognizing target cell-bound IgG via the FcγRIII (CD16) receptor. In addition, NK cells are activated by the downregulation of HLA molecules on target cells, an innate immunity evasion mechanism inherent to viral infections.

High cytokine levels, as observed in acute respiratory distress syndrome (ARDS), associated with certain coronavirus infections, further activate NK cells. Since NK cells do not act through HLA-complex binding, allogeneic NK cells do not induce graft versus host disease (GvHD), a common and sometimes lethal problem with allogeneic T cell transfer. Further, activation of large numbers of NK cells does not induce a cytokine storm, a severe toxicity often encountered when T cells expand rapidly. Activated NK cells also have the ability to bridge to the adaptive immune system and induce long term memory (O'Sullivan et al. (2015) Immunity 43:634-645).

### SUMMARY

Natural Killer (NK) cells can be used as a therapeutic for patients testing positive for infection with a virus, such as a coronavirus, as they exhibit non-HLA-dependent toxicity toward virus infected cells. The NK cells can be cells of a cell line, such as the highly cytotoxic KHYG-1 line, where dosing of the cells can be titrated according to the subject's condition or response to treatment. The cells of the NK cell line can be dosed one or multiple times, as the cells are regenerative and do not require harvesting from a donor, and the cells can be irradiated prior to administration such that they do not replicate in the subject's body. The cells may be frozen, before or after irradiation, and thawed prior to use.

The present disclosure provides methods for treating or preventing a viral infection in a subject, where the methods include administering to the subject NK cells in an amount effective to reduce, reduce the pathology of, or prevent, the viral infection in the subject. The NK cells may be primary cells, and may be allogeneic primary cells, or may be cells of a natural killer (NK) cell line. In some embodiments, the subject is treated by administration of cells of NK cell line KHYG-1. In various embodiments, the cells are not genetically engineered. In various preferred embodiments, the cells are irradiated prior to administering the cells to the patient. The subject may be administered one or more additional therapeutics, before, after, or concurrent with administration of NK cells, including but not limited to one or more antiviral compounds, one or more antibiotics, one or more cytokines, one or more cytokine blockers, one or more anti-inflammatory compounds, or one or more antibodies. The viral infection in some embodiments is a coronavirus infection, and in some examples the subject is infected with or at risk of becoming infected with, SARS-CoV-2 or another coronavirus. For example, the subject may be a subject infected with a coronavirus such as SARS-CoV-2. The subject in some embodiments may be diagnosed with ARDS.

Also provided herein is a preparation of NK cells for use in a method for treating or preventing a viral infection in a subject, where the method includes administering to the subject NK cells in an amount effective to reduce, reduce the pathology of, or prevent, the viral infection in the subject. The NK cells may be primary cells, and may be allogeneic primary cells, or may be cells of a natural killer (NK) cell line. In some embodiments, the NK cells are cells of NK cell line KHYG-1. In various embodiments, the cells are not genetically engineered. In various preferred embodiments, the cells are irradiated cells, e.g., cells that are incapable of replicating. The NK cells of the preparation may be frozen and the preparation may optionally include one or more cryoprotective agents. The method may include administering one or more additional therapeutics, before, after, or concurrent with administration of the NK cells, including but not limited to one or more antiviral compounds, one or more antibiotics, one or more cytokines, one or more cytokine blockers, one or more anti-inflammatory compounds, or one or more antibodies. The viral infection in some embodiments is a coronavirus infection, and in some examples the subject is infected with or at risk of becoming infected with SARS-CoV-2 or another coronavirus. For example, the subject may be a subject infected with SARS-CoV-2. The subject in some embodiments may be diagnosed with ARDS.

The present disclosure further provides methods of treating a patient diagnosed with ARDS where the method includes administering NK cells to the subject to reduce one or more symptoms associated with ARDS, including, for example, any of shortness of breath, rapid breathing, low PaO₂/FiO₂ ratio, and low SpO₂ (oxygen saturation). The method can further include administering one or more additional therapeutic compounds or formulations before, after, or concurrent with administration of NK cells, including but not limited to one or more antiviral compounds, one or more antibiotics, one or more cytokines, one or more cytokine blockers, one or more anti-inflammatory compounds, or one or more antibodies. In some embodiments the subject treated with NK cells is further administered one or more anti-inflammatory compositions or formulations, including but not limited to a compositions or formulation that comprises a corticosteroid, a non-steroidal anti-inflammatory drug (NSAID), or colchicine.

The present disclosure further provides methods of treating a subject diagnosed with ARDS where the method includes administering NK cells to the subject to reduce one or more symptoms associated with ARDS, including, for example, any of shortness of breath, rapid breathing, low PaO₂/FiO₂ ratio, and low SpO₂ (oxygen saturation). The subject can be infected with a virus, such as, for example, an influenza virus or coronavirus. In some embodiments, the virus is SARS-CoV-2. The method can further include administering one or more additional therapeutic compounds or formulations before, after, or concurrent with administration of NK cells, including but not limited to one or more antiviral compounds, one or more antibiotics, one or more cytokines, one or more cytokine blockers, one or more anti-inflammatory compounds, or one or more antibodies. For example, in some embodiments the subject treated with NK cells is further administered one or more anti-inflammatory compositions or formulations, including but not limited to a compositions or formulation that comprises a corticosteroid, a non-steroidal anti-inflammatory drug (NSAID), or colchicine. The NK cells administered to a subject exhibiting ARDS can be primary NK cells, and may be allogeneic primary NK cells, or can be cells of an NK cell line. In some embodiments, the NK cell line is KHYG-1. In various preferred embodiments, NK cells of an NK cell line are irradiated prior to administering the cells to the patient. In various embodiments, the NK cells are not genetically engineered.

Also provided herein is a preparation of NK cells for use in a method for treating a patient diagnosed with ARDS where the method includes administering NK cells to the subject to reduce one or more symptoms associated with ARDS, including, for example, any of shortness of breath, rapid breathing, hypoxemia, low PaO₂/FiO₂ ratio, and low SpO₂ (oxygen saturation). The method can further include administering one or more additional therapeutic compounds or formulations before, after, or concurrent with administration of NK cells, including but not limited to one or more antiviral compounds, one or more antibiotics, one or more cytokines, one or more cytokine blockers, one or more anti-inflammatory compounds, or one or more antibodies. In some embodiments the subject treated with NK cells is further administered one or more anti-inflammatory compositions or formulations, including but not limited to a compositions or formulation that comprises a corticosteroid, a non-steroidal anti-inflammatory drug (NSAID), or colchicine. In various embodiments, a subject exhibiting ARDS is a patient infected with a virus. In some embodiments, the virus is a coronavirus, for example, SARS-CoV-2. The NK cells administered to a subject exhibiting ARDS can be primary NK cells, and may be allogeneic primary NK cells, or can be cells of an NK cell line. In some embodiments, the NK cell line is KHYG-1. In various preferred embodiments, NK cells of an NK cell line are irradiated prior to administering the cells to the patient. In various embodiments, the NK cells are not genetically engineered.

In some embodiments of the methods provided herein, the NK cells to be administered to a subject exhibiting ARDS or having or at risk of contracting a viral infection, such as a coronavirus infection, may be cultured with one or more ligands or cytokines prior to administering the cells to the subject. For example, the NK cells may be cultured with the ligand 4-1BBL or a cytokine such as IL-2, IL-12, IL-15, IL-18, or IL-21, or a combination of any thereof. The cells may be administered by infusion, e.g., by infusion into the bloodstream via a vein or artery and in some embodiments may be by means of a catheter inserted into the arm or chest. In some embodiments, the NK cells can be administered intrapleurally. The number of cells administered in a single dosing can be, for example, between about 10⁴ NK cells and about 10¹⁰ NK cells, for example, can be from about 10⁴ NK cells to about 0.5×10⁵ NK cells, or from about 0.5×10⁵ NK cells to about 10⁵ NK cells, or from about 10⁵ NK cells to about 0.5×10⁶ NK cells, or from about 0.5×10⁶ NK cells to about 10⁶ NK cells, or from about 10⁶ cells to about 0.5×10⁷ NK cells, or from about 0.5×10⁷ NK cells to about 10⁷ NK cells, or from about 10⁷ cells to about 0.5×10⁸ NK cells, or from about 10⁸ cells to about 0.5×10⁹ NK cells, or from about 0.5×10⁹ NK cells to about 10⁹ NK cells, or from about 10⁹ cells to about 0.5×10¹⁰ NK cells, or from about 0.5×10¹⁰ NK cells to about 10¹⁰ NK cells.

For example, the effective amount of NK cells administered to the patient in a single dose can be about 10³ NK cells/kg of the subject, about 10⁴ NK cells/kg of the subject, about 0.5×10⁵ NK cells/kg of the subject, about 10⁵ NK cells/kg of the subject, about 2×10⁵ NK cells/kg of the subject, about 0.5×10⁶ NK cells/kg of the subject, about 10⁶ NK cells/kg of the subject, about 2×10⁶ NK cells/kg of the subject, about 0.5×10⁷ NK cells/kg of the subject, about 10⁷ NK cells/kg of the subject, about 2×10⁷ NK cells/kg of the subject, about 0.5×10⁸ NK cells/kg of the subject, about 10⁸ NK cells/kg of the subject, about 2×10⁸ NK cells/kg of the subject, about 0.5×10⁹ NK cells/kg of the subject, or about 10⁹ NK cells/kg of the subject.

A subject treated with NK cells can be administered a single dose of NK cells or multiple doses of NK cells, for example, between two and five doses, or between five and ten doses, or between ten and twenty doses, where any of the doses may be spaced at least one day apart, at least two days apart, at least three days apart, at least five days apart, at least seven days apart, at least ten days apart, at two weeks apart, at least three weeks apart, or at least four weeks apart. Administration of second, third, or subsequent doses can be based on indicators of the degree of viral infection and/or ARDS, e.g., viral load or respiratory symptoms, in the patient following a first dose.

### BRIEF DESCRIPTION OF THE DRAWINGS

FIG. 1 is a schematic showing the preparation of NK cells of a cell line for infusion into a patient infected with a virus. NK cells are expanded, irradiated, and frozen in bags for later thawing and infusion.

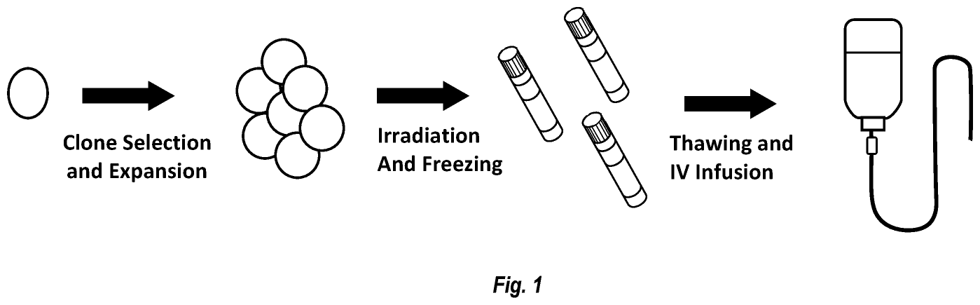

FIG. 2 provides the results of cytotoxicity assays using clonal isolates of the KHYG cell line (left graph: clonal isolates 4C12, 4F8 and 5B11; right graph: clonal isolate 3B4) as effectors and K526 cells as targets at the ratios indicated on the x axis. The lines marked with inverted triangles in the left graph and squares in the right graph represent the results of assays lacking effectors.

### DESCRIPTION OF THE INVENTION

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided throughout the specification are solely for the convenience of the reader and are not limitations of the various aspects of the disclosure, which aspects can be understood by reference to the specification as a whole.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The term “coronavirus infection” refers to a human or animal that has cells that have been infected by a coronavirus. The infection can be established by performing a detection and/or viral titration from respiratory samples, or by assaying blood-circulating coronavirus-specific antibodies. The detection in the individuals infected with coronavirus is made by conventional diagnostic methods, such as molecular biology (e.g., PCR), which are known to those skilled in the art.

The terms “natural killer” cells and “NK” cells refer to large granular lymphocytes that mediate in the innate immune response. NK cells exhibit cytolytic activity against a variety of targets via exocytosis of cytoplasmic granules containing a variety of proteins, including perforin, and granzyme proteases. Killing is triggered in a contact-dependent, non-phagocytotic process which does not require prior sensitization to an antigen. Human NK cells are characterized by the presence of the cell-surface markers CD16 and CD56, and the absence of the T cell receptor (CD3). Cell surface expression of the CD56, CD3, CD16, CD94 and other markers can be determined, for example, via FACS analysis or immune-histological staining techniques. The cytotoxicity of the NK cells can be detected using in vitro analysis of cell mediated cytotoxicity against K562 cells using the standard 4 hour ⁵¹Cr-release assay. Alternatively, the degranulation assay can be used.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine. A subject being treated using the methods herein may also be referred to as a patient.

The term “administering”, “administered” and grammatical variants refers to the physical introduction of an agent (e.g., NK cells) to a subject, using any of the various methods and delivery systems known to those skilled in the art. Exemplary routes of administration for the formulations disclosed herein include intrapleural, intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “treatment” and “treating” refer to fighting the coronavirus infection in a human or animal subject. By virtue of the administration of at least one embodiment of the compositions described herein, the viral infection rate (infectious titer) in the subject will decrease, and the virus may completely disappear from the subject. The terms “treatment” and “treating” also refers to attenuating symptoms associated with the viral infection (e.g., respiratory syndrome, kidney failure, fever, and other symptoms relating to coronavirus infections).

The terms “effective amount”, “therapeutically effective amount” or “effective dose” or related terms may be used interchangeably and refer to an amount of NK cells that when administered to a subject, is sufficient to affect a measurable improvement or prevention of a disease or disorder associated with coronavirus infection. For example, administering an effective dose sufficient to inhibit the proliferation and/or replication of the coronavirus, and/or the development of the viral infection within the subject. Therapeutically effective amounts of NK cells provided herein, when used alone or in combination with an antiviral agent, will vary depending upon the relative activity of the NK cells, and depending upon the subject and disease condition being treated, the weight and age and sex of the subject, the severity of the disease condition in the subject, the manner of administration and the like, which can readily be determined by one of ordinary skill in the art. In one embodiment, a therapeutically effective amount will depend on certain aspects of the subject to be treated and the disorder to be treated and may be ascertained by one skilled in the art using known techniques. In general, the NK cells are administered to the subject at about 10⁴ NK cells/kg of the subject, about 10⁵ NK cells/kg of the subject, about 10⁶ NK cells/kg of the subject, about 10⁷ NK cells/kg of the subject, about 10⁸ NK cells/kg of the subject, about 10⁹ NK cells/kg of the subject or 10¹⁰ NK cells/kg of the subject. The NK cells may be administered to the subject daily (e.g., once, twice, three times, or four times daily) or less frequently (e.g., 3 days apart, 5 days apart, 7 days apart, 10 days apart, 14 days apart, 17 days apart, 21 days apart, 25 days apart, 27 days apart, or 30 days apart). In addition, as is known in the art, adjustments for age as well as the body weight, general health, sex, diet, time of administration, drug interaction, and the severity of the disease may be necessary.

The terms “placental-derived NK cells”, “placenta-derived intermediate NK cells”, “placental intermediate NK cells,” “PINK,” “PINK cells,” or “PINK cells,” as used herein refers to NK cells that are obtained from placenta, e.g., human placental tissue or human placental perfusate that has been mechanically and/or enzymatically disrupted, or generated by a two-step expansion and differentiation method using hematopoietic stem cells that are recovered from placenta, as exemplified for example in U.S. Pat. No. 8,926,964. PINK cells are CD56⁺ and CD16, which can be determined using fluorescence-activated cell sorting using antibodies to CD56 and CD16. PINK cells are not obtained from umbilical cord blood or peripheral blood. In some specific embodiments, examples of PNK cells include PNK-007 cells.

The term “placental perfusate” as used herein means perfusion solution that has been passed through at least part of a placenta, e.g., a human placenta, e.g., through the placental vasculature, including a plurality of cells collected by the perfusion solution during passage through the placenta.

The term “placental perfusate cells” as used herein means nucleated cells, e.g., total nucleated cells, isolated from, or isolatable from, placental perfusate.

The term “autologous” as used herein means a population of NK cells derived from the subject to receive the treatment.

The term “allogeneic” as used herein means a population of NK cells derived from a different subject (e.g., a donor) or from multiple donors.

Viral-infected cells can evade detection and killing by T cells because the virus interferes with the cell's production of surface-expressed MHC-I complex, leading to reduced peptide presentation at the cell's surface. NK cells are part of the innate immune system and play a key role in defense against viral infection that does not require MHC-I recognition. NK cell recognition of virus-infected cells involves NKp46 activating receptors on the NK cells which bind with viral haemagglutinins on the surface of virus-infected cells.

In humans, NK cells are found in many tissues including bone marrow, blood, liver, thymus and spleen. Additionally, NK cells reside in mucosal sites such as lung, small intestine and colon. Among non-lymphoid tissues, lung contains the largest number of conventional NK cells. Many viruses infect lung airways, including coronaviruses (SARS, (MERS, and SARS-CoV-2) and influenza. NK cells respond to virus infection in several ways, including production of interferon-gamma (IFNγ) which acts distally to recruit and activate effector leukocytes. NK cells also release proinflammatory cytokines having antiviral activity. NK cells kill virus-infected cells directly by releasing cytolytic granules in close proximity to the virus-infected cells. The granules contain perforin and granzymes which permeabilizes the infected cells' membranes and triggers apoptosis via a caspase-mediated signaling pathway leading to cell death.

### Methods for Treating a Subject Having a Virus Infection

Methods are provided for treating or preventing a viral infection that include administering NK cells to the subject. The administered NK cells can reduce, reduce the pathology of, or prevent, the viral infection in the subject. The NK cells can be primary cells, for example, allogeneic primary NK cells harvested from placental tissue or peripheral blood of a donor, or may be NK cells of a cell line.

In various preferred embodiments the cells are cells of an NK cell line. The NK cells of a cell line can exhibit cytotoxicity characteristic of primary NK cells and can be irradiated prior to use such that the cells are not capable of replicating in the body of the patient. The NK cells can be frozen for storage and transport. The use of an NK cell line removes the limitations of screening of donors and testing of each cell batch and the expense and time required to select, harvest, and enrich primary cells from each donor, allowing for multiple repeated doses that can be tailored in timing and number of cells per treatment to the patient's pathology and symptoms over time.

The subject can have or be at risk of contracting a viral infection such as, for example, an influenza infection or a coronavirus infection. The virus causing the infection may be, for example, a virus that causes respiratory symptoms, such as a cough, shortness of breath, hypoxemia, low SpO₂ (oxygen saturation), or low PaO₂/FiO₂ ratio. The subject in some embodiments may have a viral infection and may be diagnosed with acute respiratory distress syndrome (ARDS). In various examples, the subject can be infected with or can be at risk of becoming infected with, a SARS virus such as MERS, SARS-CoV, SARS-CoV-2, or another coronavirus. For example, the subject may be a subject infected with SARS-CoV-2.

One or more additional therapeutic compounds may be used in addition to the one or more doses of NK cells administered to a subject having a viral infection such as a coronavirus infection. For example, one or more or any combination of: anti-inflammatory drugs, one or more antiviral compounds, one or more antibiotics, one or more cytokines, one or more cytokine blockers, one or more anti-inflammatory compounds, or one or more antibodies can be administered prior to, subsequent to, or at the same time as administration of a dose of NK cells. In some embodiments a patient can be administered, in addition to one or more doses of NK cells, one or more anti-inflammatory drugs, such as, but not limited to, a corticosteroid such as cortisone, prednisone, methylprednisone, or fludrocortisone (Prescott & Rice (2020) JAMA 324:1292-1295); an NSAID, including but not limited to ibuprofen, ketoprofen, naproxen, diclofenac potassium, or paracetamol; or colchicine. Alternatively or in addition, one or more antibodies, such as one or more antibodies that specifically binds the virus, can be administered to the subject receiving one or more doses of NK cells. For example, a patient infected with SARS-CoV-2 can be treated with one or more antibodies such as, but not limited to, bamlanivimab, etesevimab, and/or one or more other monoclonal antibodies that specifically bind a SARS-Cod′-2 protein. Alternatively or in addition, an antiviral therapeutic such as, for example, lopinavir/ritonavir, ganciclovir, oseltamivir, arbidol, Aluvia or Remdesvir can be administered before, after, or concurrent with NK cell administration.

Treatment with NK cells can reduce a viral infection in a subject which can be assessed by the viral load in a subject infected with a virus. Viral load can be determined, for example, by determining the amount of viral nucleic acid (viral genomes) in a sample from a subject, such as a blood, saliva, sputum, or swab sample (e.g., a nasal, nasopharyngeal, or throat swab). For example, for SARS-CoV-2 infection, an RT-PCR or dispersed droplet PCR (ddPCR) test using, for example, a sputum sample or nasopharyngeal swab sample can be used to determine viral load.

Treatment with NK cells can also reduce the prevalence or severity of one or more pathologies or symptoms of viral infection in a subject infected with a virus such as a coronavirus, such as but not limited to SARS-CoV-2, including, but not limited to, vomiting, diarrhea, fever, cough, headache, fatigue, muscle aches, anosmia, shortness of breath, hypoxemia, PaO₂/FiO₂, and low blood PaO₂/FiO₂ ratio.

In further embodiments prophylactic administration of NK cells, such as KHYG-1 cells, can reduce the frequency of contracting the virus or reduce the severity of the viral infection if contracted, in a population at risk of contracting the virus. Populations at risk can include, as nonlimiting examples, health care workers, nursing home or assisted living care workers, nursing home or assisted living care residents, incarcerated people, meat processing plant workers, teachers, restaurant workers, hotel workers, airline and airport workers, bus drivers, etc.

Also provided are methods of treating a subject exhibiting ARDS. ARDS can be diagnosed as lung injury with acute onset and respiratory symptoms, notably a decreased blood PaO₂/FiO₂ ratio of less than 300. Lung pathologies visible by chest imaging (radiograph or CT) are also present. (ARDS Definition Task Force. Acute respiratory distress syndrome: the Berlin definition. 425 JAMA 307, 2526-2533 (2012)). A subject exhibiting ARDS that is treated with NK cells can have a virus infection, such as a coronavirus infection, and can have a SARS-CoV-2 infection. A subject diagnosed with ARDS can be treated with NK cells using the methods provided herein, where one or multiple doses of NK cells can be administered to the patient. One or more additional therapeutic compounds or formulations, for example, one or more antibiotics, one or more antiviral compounds, one or more anti-inflammatory compounds, one or more antihistamines, one or more antigen-binding proteins, such as one or more antibodies, can be administered at the same time as, prior to, or after, delivery of a dose of NK cells. In some embodiments an antibody that binds a viral epitope is administered to the patient before, after, or at the same time as NK cells are administered to the patient. In some embodiments an antigen-binding protein that binds a cytokine is administered before, after, or at the same time as NK cells are administered to the patient. In some embodiments one or more anti-inflammatories or one or more anti-viral compounds is administered to a subject having or at risk of contracting a coronavirus infection or exhibiting ARDS. In some embodiments a patient diagnosed with ARDS can be administered NK cells, such as KHYG-1 cells, and one or more anti-inflammatory drugs, such as, but not limited to, a corticosteroid such as cortisone, prednisone, methylprednisolone, or fludrocortisone; an NSAID, including but not limited to ibuprofen, ketoprofen, naproxen, diclofenac potassium, or paracetamol; or colchicine. Alternatively or in addition, one or more antibodies, such as one or more antibodies that specifically binds the virus (e.g., an antibody that binds a coronavirus protein), can be administered to a subject having or at risk of contracting a coronavirus infection or exhibiting ARDS.

One or more additional therapeutic compounds or formulations, for example, one or more antibiotics, one or more antiviral compounds, one or more anti-inflammatory compounds, one or more antihistamines, one or more antigen-binding proteins, such as one or more antibodies, can be administered at the same time as, prior to, or after, delivery of a dose of NK cells. In some embodiments an antibody that binds a viral epitope is administered to the patient before, after, or at the same time as NK cells are administered to the patient. In some embodiments a cytokine blocker is administered before, after, or at the same time as NK cells are administered to the patient. In some embodiments one or more anti-inflammatories or one or more anti-viral compounds is administered to a subject having or at risk of contracting a coronavirus infection or exhibiting ARDS.

The present invention provides methods for treating a subject having a coronavirus infection, as well as methods for reducing viral load, methods for reducing the time to viral clearance, and/or methods for reducing the clinical outcomes of morbidity or mortality, in a subject suffering from a coronavirus infection. The methods comprise the step of administering NK cells to the subject, in an amount effective to reduce the coronavirus infection, to ameliorate the clinical course of the disease, to reduce the time to viral clearance, or to reduce morbidity or mortality in clinical outcomes. In some embodiments, the administered NK cells can be obtained or derived from placenta, cord blood or PBMCs. In further embodiments, the administered NK cells can be NK cells of a cell line, for example, the KHYG-1 cell line. In various embodiments the administered NK cells are irradiated KHYG-1 cells. In various embodiments, the cells are not genetically engineered. The subject may be administered one or more additional therapeutics, before, after, or concurrent with administration of NK cells, including but not limited to one or more antiviral compounds, one or more antibiotics, one or more cytokines, one or more cytokine blockers, one or more anti-inflammatory compounds, or one or more antibodies.

A human subject having a coronavirus infection may exhibit any one or any combination of symptoms including fever, cough, headache, fatigue, sore throat and/or gastrointestinal symptoms. A human subject having a coronavirus infection may be diagnosed as having a coronavirus infection by obtaining a respiratory sample (e.g., nasal swab), saliva sample, and/or blood sample and conducting reverse chain PCR (RT-PCR) or quantitative PCR to detect viral nucleic acid sequences or immunological testing to detect viral protein(s). In some embodiments, a viral load can be detected by measuring the titer or level of viral nucleic acid or protein in serum (e.g., blood serum). In some embodiments, the presence of viral particles or the number of viral particles or the number of viral genomes in serum can be determined using a PCR assay using oligonucleotide primers specific for the coronavirus being assayed. In one embodiment, a viral load can be detected by measuring the titer or level of virus in serum (e.g., blood serum). In one embodiment, the presence of viral particles or the number of viral particles or the number of viral genomes in serum can be determined using one or more serological assays.

An effective amount of NK cells can ameliorate (reduce) the level of detectable coronavirus (virus load) in the subject which can be assayed by RT-PCR and/or serological assays. In one embodiment, treating the subject with the NK cells can reduce the virus load by about 10-20%, or by about 20-30%, or by about 30-40%, or by about 40-50%, or by about 50-60%, or by about 60-70%, or by about 70-80%, or by about 80-90%, or by about 90% or higher levels, compared to the viral load in a subject that has not been administered an effective amount of NK cells.

An effective amount of NK cells can reduce morbidity or mortality in a population of subjects due to a coronavirus infection by about 10-20%, or by about 20-30%, or by about 30-40%, or by about 40-50%, or by about 50-60%, or by about 60-70%, or by about 70-80%, or by about 80-90%, or by about 90% or higher levels, compared to the viral load in a population of subjects that has not been administered an effective amount of NK cells.

### NK Cells

NK cells used in the methods provided herein can be a primary cells or cells of a cell line.

### NK Cell Lines

NK cell lines can be, without limitation, KYHG cells, NK92 cells, YTS cells, NK3.3 cells, NK-YS, NK-YT, NKL, NKG, MOTN-1, HANK-1, SNK-6, IMC-1, NKL cells, or other NK cell lines. An NK cell line used in the methods or compositions of the present invention can be developed for the purpose of cell therapy as set forth herein. Preferably cells of a cell line that are used in cancer therapy are irradiated prior to delivery to a patient, where irradiation prevents the cells from dividing.

In some embodiments, the methods include administering KHYG-1 NK cells to a subject, e.g., a subject infected with a virus, a subject at risk of becoming infected with a virus, or a subject diagnosed with ARDS. The virus can be a coronavirus. For example, the methods include administering KHYG-1 NK cells to a subject, e.g., a subject infected with SARS-CoV-2, a subject at risk of becoming infected with SARS-CoV-2, or a subject diagnosed with ARDS where the subject is infected with a coronavirus such as SARS-CoV-2. KHYG-1 is a highly cytotoxic cell line. The activation receptor NKp44 and its adaptor, DAP12, NKG2D, and constitutively phosphorylated ERK2 are associated with the enhanced cytotoxicity of KHYG-1. The KHYG-1 cell line mediates cytolysis by granzyme M (but not granzymes A and B) together with perforin (Suck G et al, Exp Hematol 2005). KHYG-1 cells can be cultured (e.g., in RPMI 1640 medium containing 2 mM L-Glutamine, 20% FBS, 2 mM sodium pyruvate, supplemented with 450 U/ml rhIL-2) and irradiated at 10 Gy (Suck G et al. (2006) Int J Radiat Biol). Following irradiation, the cells are allowed to recover in culture for example, for twenty-four hours, and can then be frozen or used directly.

### Primary NK Cells

Primary NK cells can be isolated from human whole blood, PBMCs (peripheral blood mononuclear cells), or buffy coat. For example, NK cells can be directly isolated or enriched from PBMCs using density gradient centrifugation. For example, NK cells can be directly isolated/enriched from PBMCs using positive magnetic enrichment for CD56+NK cells (e.g., using MACS separation technology including Whole Blood CD56 MicroBead Kit or Buffy Coat CD56 MicroBead Kit, both from Miltenyi BioTec).

In some embodiments, a magnetic depletion step can be employed to remove CD3+ T cells from PBMCs. In one embodiment, the magnetic depletion step can be employed using the MACSxpress NK Cell Isolation Kit (from Miltenyi BioTec). In some embodiments, the PBMCs can be obtained from one or more healthy human donors via leukapheresis. The depleted cells can be stimulated and expanded with irradiated autologous PBMCs in the presence of OKT3 and IL-2, for example for approximately 14 days, to generate a population of NK cells that are CD3-CD16+ CD56+.

In some embodiments, the isolated NK cells are not expanded or activated. In some embodiments, the isolated NK cells are expanded and activated.

Methods are provided for treating a subject having a coronavirus infection, including a subject exhibiting ARDS, or for prophylactically treating an asymptomatic subject, the method comprising administering to the subject a population of human NK that have been expanded and activated from human PBMCs, in an amount effective to reduce the coronavirus infection or reduce the pathological indicators of ARDS. In one embodiment, the expanded and activated NK cells are isolated NK cells.

One skilled in the art will recognize there are many different procedures for expanding and activated a population of isolated NK cells. In one embodiment, the isolated NK cells are placed in an expansion/activation reaction mixture with any one or any combination of 2-3 cytokines, including IL-2, IL12, IL21, IL15 and/or IL18, under conditions that are suitable for expanding and activating the isolated NK cells. In one embodiment, the expansion/activation reaction mixture also include any one or any combination of 2-4 of the following agents: anti-NKp46 antibody, B7-H6-Fc, anti-NKp30 antibody and/or 4-1BBL-Fc.

In one embodiment, the cells of an NK cell line, human peripheral blood, or human PBMCs are (i) subjected (e.g., in a closed system) to a growth medium supplemented with serum, interleukin-2 (IL-2) and anti-CD3 antibodies; and (ii) expanded within the system with agitation and heating until at least 35% of the expanded cell population comprises activated NK cells and NK-like T cells, and the expanded cells exhibit an increased cytotoxicity as determined by in Vitro cytotoxicity tests. In one embodiment; the serum can be human serum or autologous serum. The medium can be supplemented with about 50 to about 1500 U/ml IL-2, about 1 to about 50 ng/ml anti-CD3-antibodies and about 1 to about 40% serum. In one embodiment, the human PBMCs can be expanded and activated according to the description in European patent No. 2411507, entitled “Expansion of NK Cells”.

Agitation and heating can be performed under the following conditions: a temperature of about 36-40° C.; a CO₂, concentration of about 4.7-5.1%; and gentle rocking at a rate and angle permitting the cells to adhere to the surface of the closed cell system. For example, rocking can be performed at a g rate of about 4-8/min and at an angle of about 4-8′.

Expansion can be performed until the total number of cells has expanded at least about 10-fold or until at least about 50% of the expanded cell population comprises activated NK cells. The concentration of cells initially added to the closed cell system can be about 0.5×10⁶ to about 2×10⁶/ml growth medium.

In some embodiments, the method further comprises the step of adding per day a volume of the medium supplemented with serum and IL-2 corresponding to about 50% of the total culture volume and discarding about the same volume of growth medium/day from the closed cell system, wherein the step is performed when the total cell density has increased with at least about 50% of the initial cell density.

In some embodiments, the step is performed when the total cell density has increased with at least about 300 from the initial cell density, but wherein about 75% of the total culture volume is exchanged. Alternatively, the step is performed when the total cell density has increased with at least about 500% from the initial cell density, but wherein about 100% of the total culture volume is exchanged.

In some embodiments, freely combinable with one or more of the above disclosed embodiments, the cells are incubated for at least about 10 days.

In some embodiments, the closed cell system is a pre-sterile bag or a bioreactor. In some embodiments, the closed system is a cell-growth chamber system consisting of a central cell culture bag wherein cells can be efficiently and quickly proliferated ex vivo, without any further steps of passage.

In some embodiments, the resulting NK cells exhibit a phenotype CD3-CD56⁺.

In some embodiments, the NK cells exhibit an increased cytotoxicity, as determined by in vitro cytotoxicity tests, compared to cells expanded in flasks.

In some embodiments, at least 35-45%, or at least 45-55%, or at least 55-65%, or at least 65-75%, or 75% or high levels of the expanded NK cells are activated NK cells. In some embodiments, the activated NK cells have received an activating signal and are capable of cells with deficiencies in WIC class I expression.

A subject having a coronavirus infection, including a subject diagnosed with ARDS, can be administered a population of human placenta-derived NK cells in an amount effective to reduce the coronavirus infection. For example, placental-derived INK cells can be isolated from umbilical cord blood or placental perfusate or NK cells can be differentiated from CD34⁺ hematopoietic stem cells recovered from umbilical cord blood or placental perfusate.

Human placenta-derived NK cells can be prepared by: (a) culturing hematopoietic stem or progenitor cells in a first medium comprising a stem cell mobilizing agent and thrombopoietin (Tpo) to produce a first population of cells; (b) culturing, the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking a stem cell mobilizing agent and LMWH, to produce a third population of cells. The third population of cells can comprise NK cells that are CD56+, CD3−, CD16− or CD16+, and CD94+ or CD94−, where at least 80% of the INK cells can be viable. Human placenta-derived NK cells are prepared as described in U.S. published application No. 2019/0093081, entitled “Placental-Derived Intermediate Natural Killer (PINK) Cells for Treatment of Glioblastoma”.

For example, Tpo can be present in the first medium at a concentration of about 1 rig/mL to 50 ng/mL or at a concentration of about 20 ng/mL to 30 rig and can be present in the second medium at a concentration of about 1 ng/mL to 50 ng/mL, for example, from about 10 ng/mL to 30 ng/mL. The stem cell mobilizing agent of the first medium can be an aryl hydrocarbon receptor inhibitor such as StemRegenin-1 (SR-1) (4-(2-(2-(benzo[b]thiophen-3-yl)-9-isopropyl-9H-purin-6-ylamino)ethyl)phenol), resveratrol, or the compound CH:223191 (1-Methyl-N-[2-methyl-4-[2-(2-methylphenyl)diazenyl]phenyl-1H-pyrazole-5-carboxamide]. In another example, the stem cell mobilizing agent is a pyrimido(4,5-b)indole derivative, such as any described in US 20190093081, incorporated by reference herein in its entirety.

IL-2 can be present in the third medium at a concentration of about 10 U/mL to 10,000 U/mL, along with IL-15 at a concentration of about 1 ng/mL to 50 ng/mL. In one embodiment, the IL-2 is present in the third medium at a concentration of about 300 U/mL to 3,000 U/mL and the IL-15 is present in the third medium at a concentration of about 10 ng/mL to 30 ng/mL. Ire one embodiment, the IL-2 is present in the third medium at a concentration of about 1,000 U/mL and the IL-15 is present in the third medium at a concentration of about 20 ng/mL.

The first medium can additionally comprise one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In some embodiments, the first medium comprises each of LMWH, Flt-3L, SCF, IL-7, G-CSF, and GM-CSF. For example, in the first medium the LMWH can be present at a concentration of about 1 U/mL to 10 U/mL; the Fit-3L can be present at a concentration of about 1 ng/mL to 50 ng/mL, the SCF can be present at a concentration of about 1 ng/mL to 50 ng/mL; the IL-6 can be present at a concentration of about 0.01 ng/mL to 0.1 ng/mL; the IL-7 can be present at a concentration of about 1 ng/mL to 50 ng/mL; the G-CSF can be present at a concentration of about 0.01 ng/mL, to 0.50 ng/mL; and the GM-CSF can be present at a concentration of about 0.005 ng/mL to 0.1 ng/mL. In some embodiments, in the first medium the LMWH is present in the first medium at a concentration of about 4 U/mL; to 5 U/mL; the Flt-3L is present at a concentration of about 20 ng/mL to 30 ng/mL; the SCF is present at a concentration of about 20 ng/mL to 30 ng/mL; the IL-6 is present at a concentration of about 0.04 to 0.06 ng/mL, the IL-7 is present at a concentration of about 20 ng/mL to 30 ng/mL; the G-CSF is present at a concentration of about 0.20 ng/mL to 0.30 ng/mL; and the GM-CSF is present at a concentration of about 0.005 ng/mL to 0.5 ng/mL. In one embodiment, in the first medium the LMWH is present in the first medium at a concentration of about 4.5 U/mL; the Fit-3L is present at a concentration of about 25 ng/mL; the SCE is present at a concentration of about 27 ng/mL, the IL-6 is present at a concentration of about 0.05 ng/mL; the IL-7 is present at a concentration of about 25 ng/mL, the G-CSF is present at a concentration of about 0.25 ng/mL; and the GM-CSF is present at a concentration of about 0.01 ng/mL.

In further examples, the second medium additionally comprises one or more of LMWH, Flt-3, SCF, IL-6, IL-7, G-CSF, and GM-CSF. In some examples, the second medium additionally comprises each of LMWH, Flt-3, SCF, IL-6, IL-7, G-CSF, and GM-CST. In one embodiment, in the second medium the LMWH is present at a concentration of about 1 U/mL to 10 U/mL; the Fit-3L is present at a concentration of about 1 ng/mL to 50 ng/mL; the SCE is present at a concentration of about 1 ng/mL to 50 ng/mL; the IL-6 is present at a concentration of about 0.01 ng/mL to 0.1 ng/mL; the IL-7 is present at a concentration of about 1 ng/mL to 50 ng/mL; the G-CSF is present at a concentration of about 0.01 ng/mL to 0.50 ng/mL; and the GM-CSF is present at a concentration of about 0.005 ng/mL to 0.1 ng/mL. In one embodiment, in the second medium the LMWH is present in the second medium at a concentration of about 4 U/mL to 5 U/mL; the Flt-3L is present at a concentration of about 20 ng/mL to 30 ng/mL; the SCF is present at a concentration of about 20 ng/mL to 30 ng/mL; the IL-6 is present at a concentration of about 0.04 ng/mL to 0.06 ng/mL; the IL-7 is present at a concentration of about 20 ng/mL to 30 ng/mL; the G-CSF is present at a concentration of about 0.20 ng/mL to 0.30 ng/mL; and the GM-CSF is present at a concentration of about 0.005 ng/mL to 0.5 ng/mL. In one embodiment, in the second medium the LMWH is present in the second medium at a concentration of about 4.5 Unit; the Flt-3L is present at a concentration of about 25 ng/mL; the SCE is present at a concentration of about 27 ng/mL, the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL.

In some embodiments, the third medium additionally comprises one or more of SCF, IL-6, G-CSF, or GM-CSF. In one embodiment, the third medium comprises each of SCF, IL-6, IL-7, G-CSF, and GM-CSF. In some embodiments, in the third medium the SCF is present at a concentration of about 1 ng/mL to 50 ng/mL; the IL-6 is present at a concentration of about 0.01 ng/mL to 0.1 ng/mL; the IL-7 is present at a concentration of about 1 ng/mL to 50 ng/mL, the G-CSF is present at a concentration of about 0.01 ng/mL to 0.50 ng/mL; and the GM-CSF is present at a concentration of about 0.005 ng/mL to 0.1 ng/mL. In one embodiment, in the third medium the SCF is present at a concentration of about 20 ng/mL to 30 ng/mL; the is present at a concentration of about 0.04 to 0.06 ng/mL; the IL-7 is present at a concentration of about 20 ng/mL to 30 ng/mL; the G-CSF is present at a concentration of about 0.20 ng/mL to 0.30 ng/mL; and the GM-CSF is present at a concentration of about 0.005 ng/mL to 0.5 ng/mL. In one embodiment, in the third medium the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL, and the GM-CSF is present at a concentration of about 0.01 ng/mL.

In some examples, any of the first medium, second medium and/or third medium comprises 1-20% human serum-AB. In one embodiment, any of the first medium, second medium and/or third medium comprises 5%-15%, or 10% human serum-AB. In some embodiments, any of the first medium, second medium and/or third medium comprises 2-mercaptoethanol. In some embodiments, any of the first medium, second medium and/or third medium comprises gentamycin.

In some examples, the method comprises culturing the hematopoietic stem cells (e.g., from human placenta) in the first medium for 7-13 days, for 8-12 days, or for about 10 days. In some embodiments, the method comprises culturing the first population of cells in the second medium for 2-b days, for 3-5 days, or for about 4 days. In some embodiments, the method comprises culturing the second population of cells in the third medium for 10-30 days, for 15-25 days, or for about 21 days.

In one embodiment, the culturing in the first medium, second medium and third medium are all conducted under static culture conditions. In one embodiment, the culturing in at least one of the first medium, second medium or third medium are conducted in a spinner flask.

In one embodiment, the culturing in the first medium and the second medium is conducted under static culture conditions, and the culturing in the third medium is done in a spinner flask.

In one embodiment, the hematopoietic cells are initially inoculated into the first medium from 1×10⁴ to 1×10⁵ cells/mL, or at about 3×10⁴ cells/mL. In one embodiment, the first population of cells are initially inoculated into the second medium from 5×10⁴ to 5×10⁵ cells/mL, or at about 1×10⁵ cells/mL. In one embodiment, the second population of cells is initially inoculated into the third medium from 1×10⁵ to 5×10⁶ cells/mL or from 1×10⁵ to 1×10⁶ cells/mL, at about 5×10⁵ cells/mL, or at about 3×10⁵ cells/mL.

In one embodiment, the method produces at least 5000-, 10,000-, 50,000-, or 75,000-fold more NK cells as compared to the number of hematopoietic stem cells initially inoculated into the first medium. In one embodiment, the method produces NK cells that comprise at least 20%, at least 40%, at least 60%, or at least 80% CD56+ CD3− NK cells.

In one embodiment, the NK cells exhibit at least 20% cytotoxicity against K562 cells when the INK cells and the K562 cells are co-cultured in vitro at a ratio of 10:1. In one embodiment, the NK cells exhibit at least 35%, at least 45%, at least 60%, or at least 75% cytotoxicity against the K562 cells.

In one embodiment, viability of the NK cells is determined by 7-aminoactinomycin D (7AAD) staining. In one embodiment, viability of the NK cells is determined by annexin-V staining. In one embodiment, viability of the NK cells is determined by both 7-AAD staining and annexin-V staining. In one embodiment, viability of the NK cells is determined by trypan blue staining.

In one embodiment, the human placenta-derived NK cells are prepared by a method comprising the additional step of cryopreserving the population of cells after step (c). In one embodiment, the cryopreserved cell population is administered to the subject within about twenty-four, sixteen, twelve, eight, six, four, or two hours after thawing. In one embodiment, the human placenta-derived INK cells are not cryopreserved.

In one embodiment, the subject is administered the placental-derived NK cells at about 10⁴ NK cells/kg of the subject, 10⁵ NK cells/kg of the subject, 10⁶ NK cells/kg of the subject, 10⁷ NK cells/kg of the subject, 10⁸ NK cells/kg of the subject, 10⁹ NK cells/kg of the subject or 10¹⁰ NK cells/kg of the subject.

### Example

### Cytotoxicity of KHYG-1 NK Cells.

Cell line KHYG-1 was derived from the blood of a patient with NK cell leukemia (Yagita M et al, Leukemia 2000). The cells display NK cell activity and IL-2-dependent proliferation in vitro. Epstein-Barr virus (EBV) DNA was not detected in KHYG-1 cells. We have confirmed that KHYG-1 cells do not express detectable levels of ACE2.

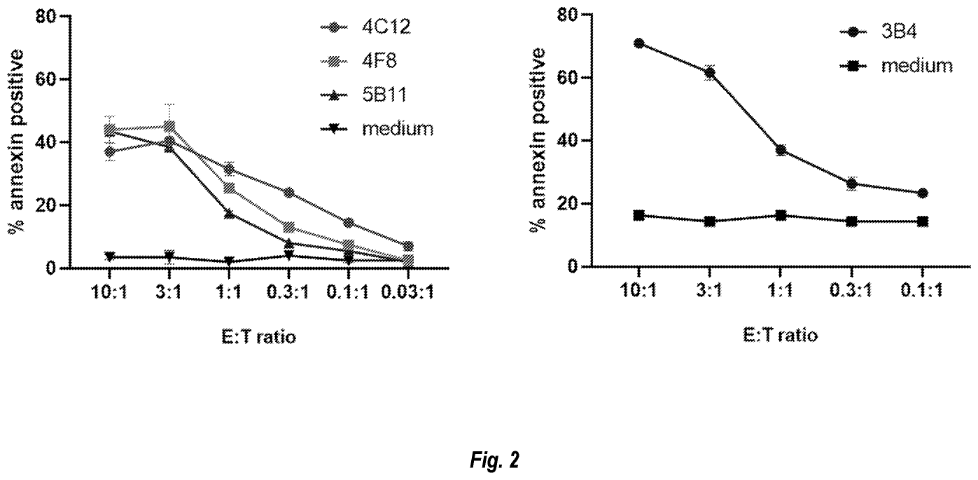

Several clonal isolates of the KHYG-1 cell line were tested in cytotoxicity assays for their ability to kill K526 cells. FIG. 2 provides the results of these assays, where each of the KHYG-1 clones was tested at effector:target cell ratios ranging from 0.1:1 to 10:1. Labeled annexin V was used to detect apoptotic cells which were analyzed by flow cytometry (e.g., Oyer et al. (2015) Biol Blood Marrow Transpl 21: 632-639). The data show that each of the isolated clones was highly cytotoxic against K526 target cells.

What is claimed:

1. A method for treating a coronavirus infection in a subject, comprising administering to the subject cells of a natural killer (NK) cell line in an amount effective to reduce the coronavirus infection in the subject.

2. The method of claim 1, wherein the NK cell line is KHYG-1.

3. The method of claim 1 or 2, wherein the cells are irradiated prior to administering.

4. The method of any of claims 1-3, wherein the cells are not genetically modified.

5. The method of any of claims 1-4, wherein prior to administering the cells are cultured with one or more ligands or cytokines.

6. The method of claim 5, wherein prior to administering the cells are cultured with one or more of 4-1BBL, IL-2, IL-12, IL-15, IL-18, or IL-21.

7. The method of any of claims 1-6, wherein the cells are administered by infusion.

8. The method of any of claims 1-6, wherein the cells are administered intrapleurally.

9. The method of any of claims 1-8, wherein the coronavirus infection is a SARS-CoV-2 infection.

10. The method of any one of the preceding claims, wherein the subject is administered a single dose of NK cells.

11. The method of any of claims 1-9, wherein the subject is administered at least two doses of NK cells.

12. The method of claim 11, wherein the subject is administered 2-10 doses of NK cells.

13. The method of claim 11, wherein the subject is administered 10-15 doses of NK cells.

14. The method of any one of claims 11-13, wherein the doses of NK cells are administered to the subject 1 day apart, 2 days apart, 3 days apart, 3 days apart, 5 days apart, 6 days apart, 7 days apart, 8 days apart, 9 days apart, 10 days apart, 12 days apart, 14 days apart, 17 days apart, 21 days apart, 25 days apart, 27 days apart, or 30 days apart.

15. The method of any one of the preceding claims, wherein the number of cells administered is about 0.5×10⁶ NK cells, about 1×10⁶ NK cells, about 0.5×10⁷ NK cells, about 1×10⁷ NK cells, about 2×10⁷ NK cells, about 0.5×10⁸ NK cells, about 1×10⁸ NK cells, about 0.5×10⁹ NK cells, about 1×10⁹ NK cells, or about 0.5×10¹⁰ NK cells per dose.

16. The method of any one of the preceding claims, wherein the number of cells administered is about 10³ NK cells/kg of the subject, about 10⁴ NK cells/kg of the subject, about 5×10⁴ NK cells/kg of the subject, about 10⁵ NK cells/kg of the subject, about 2×10⁵ NK cells/kg of the subject, about 5×10⁵ NK cells/kg of the subject, about 10⁶ NK cells/kg of the subject, about 2×10⁶ NK cells/kg of the subject, about 5×10⁶ NK cells/kg of the subject, about 10⁷ NK cells/kg of the subject, about 2×10⁷ NK cells/kg of the subject, about 5×10⁷ NK cells/kg of the subject, about 10⁸ NK cells/kg of the subject, about 5×10⁸ NK cells/kg of the subject, or about 10⁹ NK cells/kg of the subject.

17. The method of any of the previous claims, wherein the NK cells are administered to the subject via a controlled drug delivery device.

18. The method of any of the preceding claims, wherein the subject is further administered one or more additional therapeutics, before, after, or simultaneous with one or more doses of NK cells.

19. The method of claim 18, wherein the subject is further administered one or more anti-inflammatory drugs, one or more cytokines, one or more cytokine blockers, one or more antibiotics, one or more antiviral agents, or one or more antibodies.

20. The method of claim 19, wherein the subject is further administered one or more antibodies.

21. The method of claim 20, wherein the subject is further administered one or more antibodies that specifically binds a coronavirus protein.

22. The method of claim 19, wherein the subject is further administered one or more anti-inflammatory drugs.

23. The method of claim 22, wherein the subject is further administered a corticosteroid, an NSAID, or colchicine.

24. The method of claim 22, wherein the subject is further administered colchicine.

25. The method of claim 19, wherein the subject is further administered an anti-viral agent.

26. The method of claim 25, wherein the wherein the anti-viral agent comprises Aluvia or Remdesvir (GS-5734).

27. A method for preventing a coronavirus infection in a subject, comprising administering to the subject cells of a natural killer (NK) cell line in an amount effective to prevent the coronavirus infection in the subject.

28. The method of claim 27, wherein the NK cell line is KHYG-1.

29. The method of claim 27 or 28, wherein the cells are irradiated prior to administering.

30. The method of any of claims 27-29, wherein the cells are not genetically modified.

31. The method of any of claims 27-30, wherein prior to administering the cells are cultured with one or more ligands or cytokines.

32. The method of claim 31, wherein prior to administering the cells are cultured with one or more of 4-1BBL, IL-2, IL-12, IL-15, IL-18, or IL-21.

33. The method of any of claims 27-32, wherein the cells are administered by infusion.

34. The method of any of claims 27-32, wherein the cells are administered intrapleurally.

35. The method of any of claims 27-34, wherein the coronavirus infection is a SARS-CoV-2 infection.

36. The method of any one of the preceding claims, wherein the subject is administered a single dose of NK cells.

37. The method of any of claims 1-9, wherein the subject is administered at least two doses of NK cells.

38. The method of any one of the preceding claims, wherein the number of cells administered is about 0.5×10⁶ NK cells, about 1×10⁶ NK cells, about 0.5×10⁷ NK cells, about 1×10⁷ NK cells, about 2×10⁷ NK cells, about 0.5×10⁸ NK cells, about 1×10⁸ NK cells, about 0.5×10⁹ NK cells, about 1×10⁹ NK cells, or about 0.5×10¹⁰ NK cells per dose.

39. The method of any one of the preceding claims, wherein the number of cells administered is about 10³ NK cells/kg of the subject, about 10⁴ NK cells/kg of the subject, about 5×10⁴ NK cells/kg of the subject, about 10⁵ NK cells/kg of the subject, about 2×10⁵ NK cells/kg of the subject, about 5×10⁵ NK cells/kg of the subject, about 10⁶ NK cells/kg of the subject, about 2×10⁶ NK cells/kg of the subject, about 5×10⁶ NK cells/kg of the subject, about 10⁷ NK cells/kg of the subject, about 2×10⁷ NK cells/kg of the subject, about 5×10⁷ NK cells/kg of the subject, about 10⁸ NK cells/kg of the subject, about 5×10⁸ NK cells/kg of the subject, or about 10⁹ NK cells/kg of the subject.

40. The method of any of the preceding claims, wherein the subject is further administered one or more additional therapeutics, before, after, or simultaneous with one or more doses of NK cells.

41. The method of claim 19, wherein the subject is further administered one or more antibodies.

42. The method of claim 41, wherein the subject is further administered one or more antibodies that specifically binds a coronavirus protein.

43. The method of claim 40, wherein the subject is further administered an anti-viral agent.

44. The method of claim 43, wherein the anti-viral agent comprises Aluvia or Remdesvir (GS-5734).

45. A method for preventing and/or treating acute respiratory distress syndrome (ARDS) in a subject, comprising administering to the subject natural killer (NK) cells in an amount effective to reduce the severity of or prevent ARDS in the subject.

46. The method of claim 45, wherein the NK cells are primary NK cells.

47. The method of claim 45, wherein the NK cells are cells of an NK cell line.

48. The method of claim 47, wherein the NK cell line is KHYG-1.

49. The method of claim 47 or 48, wherein the cells are irradiated prior to administering.

50. The method of any of claims 47-49, wherein the cells are not genetically modified.

51. The method of any of claims 47-50, wherein prior to administering the cells are cultured with one or more ligands or cytokines.

52. The method of claim 31, wherein prior to administering the cells are cultured with one or more of 4-1BBL, IL-2, IL-12, IL-15, IL-18, or IL-21.

53. The method of any of claims 47-52, wherein the cells are administered by infusion.

54. The method of any of claims 47-52, wherein the cells are administered intrapleurally.

55. The method of any of claims 47-54, wherein the coronavirus infection is a SARS-CoV-2 infection.

56. The method of any one of claims 45-55, wherein the subject is administered a single dose of NK cells.

57. The method of claim 56, wherein the subject is administered at least two doses of NK cells.

58. The method of any of claims 45-57, wherein the number of cells administered is about 0.5×10⁶ NK cells, about 1×10⁶ NK cells, about 0.5×10⁷ NK cells, about 1×10⁷ NK cells, about 2×10⁷ NK cells, about 0.5×10⁸ NK cells, about 1×10⁸ NK cells, about 0.5×10⁹ NK cells, about 1×10⁹ NK cells, or about 0.5×10¹⁰ NK cells per dose.

59. The method of any one of claims 45-58, wherein the number of cells administered is about 10³ NK cells/kg of the subject, about 10⁴ NK cells/kg of the subject, about 5×10⁴ NK cells/kg of the subject, about 10⁵ NK cells/kg of the subject, about 2×10⁵ NK cells/kg of the subject, about 5×10⁵ NK cells/kg of the subject, about 10⁶ NK cells/kg of the subject, about 2×10⁶ NK cells/kg of the subject, about 5×10⁶ NK cells/kg of the subject, about 10⁷ NK cells/kg of the subject, about 2×10⁷ NK cells/kg of the subject, about 5×10⁷ NK cells/kg of the subject, about 10⁸ NK cells/kg of the subject, about 5×10⁸ NK cells/kg of the subject, or about 10⁹ NK cells/kg of the subject.

60. The method of any one of claims 45-59, wherein the subject is further administered one or more additional therapeutics, before, after, or simultaneous with one or more doses of NK cells.

61. The method of claim 60, wherein the subject is further administered one or more antibodies.

62. The method of claim 61, wherein the subject is further administered one or more antibodies that specifically binds a coronavirus protein.

63. The method of claim 60, wherein the subject is further administered an anti-inflammatory agent.

64. The method of claim 63, wherein the anti-viral agent comprises a corticosteroid, an NSAID, or colchicine.
